(19) United States
(12) Patent Application Publication
(10) Pub. No.: US 2004/0171578 A1
(43) Pub. Date: Sep. 2, 2004

(54) Use of corrinoids in the treatment of skin diseases

(76) Inventor: Karsten Klingelholler, Wuppertal (DE)

Correspondence Address: Friedrich Kueffner, Suite 910, 317 Madison Avenue, New York, NY, 10017, US

(21) Appl. No.: 10/480,873

(22) PCT Filed: Jun. 26, 2002
(86) PCT No.: PCT/EP02/07067

(30) Foreign Application Priority Data
Jun. 28, 2001 (DE) 10130846.9

(51) Int. Cl. A61K 31/714

(52) U.S. Cl. 514/52

(57) ABSTRACT

The use of compounds of the formula I

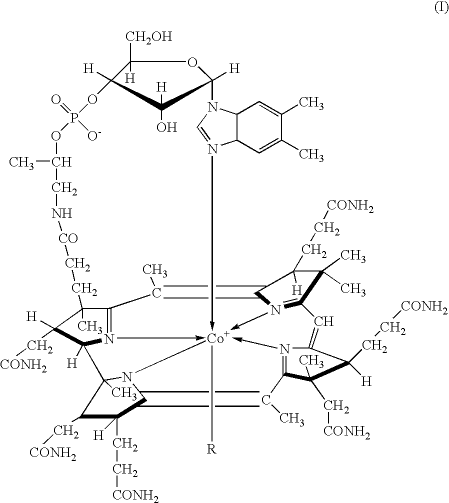

in which R stands for CN, OH, CH₃ or H₂O, for building up and improving the resistance of the skin is claimed. The compounds are suitable in particular for preventive treatment in cases of predisposition to chronic skin diseases, for the prevention and treatment of allergic skin reactions, for the treatment of acute skin injuries, for example burns and wounds, as well as in aging of the skin, and in the treatment of degenerate cells, such as in cancers, benign and/or malignant tumours.

The invention relates to a novel use of corrinoids for the treatment of acute and chronic skin diseases and skin injuries and for the prevention of certain skin diseases.

The treatment of skin diseases, in particular of chronic skin diseases, represents a major problem in medicine, because they can be remedied only to a very limited degree. The treatment of these diseases in many cases affords the patients only slight relief, and in many cases no successful cure can be observed at all. In addition, a great many of the active substances used, for example cortisone, have substantial side effects.

Because of the substantial side effects of the active substances used, they are employed only in acute cases. There is at present no preventive treatment available for predisposition to chronic skin diseases, as is the case in particular with neurodermatitis and psoriasis.

Great attention is also being paid to the prevention of other than chronic skin diseases, and of possible skin injuries, and also to skin care. To this end, skin care products and corresponding pharmaceutical preparations are used containing different ingredients and active substances, for example moisturizing substances, smoothing substances, etc.

For the treatment of skin injuries, for example burns, there are cooling gels available on the market which substantially provide pain relief but which do not generally promote the skin-healing process.

Now that a connection has been established between exposure to intense sunlight and the development of skin cancer, many people are protecting themselves against overexposure to the sun by using what are called sunscreen agents, which contain UV filters or absorbers. The UV absorbers or UV filters are organic substances which are able to absorb electromagnetic radiation in the UV range. The molecules pass into an energy-excited state. On return to their basic state, the absorbed energy is released again completely, albeit in a form which does not damage the skin or causes less damage to the skin, for example in the form of heat or light of greater wavelength and lower frequency.

A further area of treatment of skin injuries concerns wound-healing problems and scar formation. Wound-healing problems, i.e. the delayed or impaired healing of open wounds, are often associated with considerable inconvenience since open wounds can be painful and can also become inflamed. Even the scars left behind after normal wound-healing are undesirable for aesthetic reasons, especially if they are present on areas of the skin which are generally not covered by clothing, for example the face and hands.

The object of the present invention is to find an active substance which is effective for preventive treatment in cases of predisposition to chronic skin diseases, for the prevention and treatment of allergic skin reactions, for example sun allergies (dermatitis solaris, dermatitis vernalis aurium, dermatitis bullosa pratensis); for the treatment of acute skin injuries, for example burns, including sunburn and other radiation injuries (phototoxic reactions, photoallergic reactions, skin damage caused by ionizing radiation (acute and chronic radiodermatitis)) and for the treatment of skin injuries such as wounds and the resulting scars, and in striae cutis distensae and aging of the skin, for example wrinkling, including cellulitis, and cold injuries of the skin (congelation), skin injuries caused by the action of acids and alkalis, phacomatoses (Recklinghausen's disease), dermato-mycoses, polymorphous photodermatoses, ichthyoses, and in the treatment of degenerate cells, such as in cancers, benign and/or malignant tumours, and which active substance has as far as possible no side effects. In the preventive treatment of chronic skin diseases, which should if possible be carried out in infancy, it is especially necessary that the active substance has no side effects.

The present invention relates to the use of corrinoids of the formula I

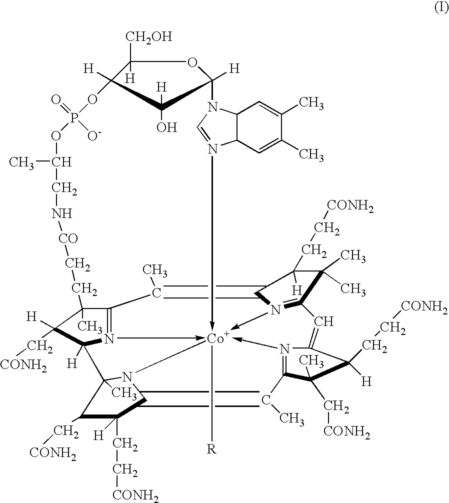

in which R stands for CN, OH, CH₃ or H₂O, for building up and improving the resistance of the skin.

The corrinoids of the formula I are also known as cobalamins or vitamin B₁₂. In medicine, they are administered orally, subcutaneously or intravenously, in the form of tablets or injection solution, respectively, for example for the treatment of pernicious anemia. Their use for the treatment of skin diseases is disclosed in European Patent 705 102. In addition to the cobalamins, further derivatives which may be mentioned here as being suitable for the use according to the invention are the cobamides, cobamic acids, cobinamides, cobinic acids, cobyric acids and cobyrinic acids.

Compounds of the formula I are preferably used for the prevention of chronic skin diseases, in particular of inflammatory and hyperproliferative skin diseases, and cutaneous manifestations of immunologically induced diseases, for example psoriasis, atopic dermatitis, contact dermatitis and other eczematous dermatitides, seborrheic dermatitis, neurodermatitis, decubitus ulcers, lichen planus, pemphigus, bullous pemphigoid, epidermolysis bullosa, urticaria, angioedemas, vasculitis, erythemas, cutaneous eosinophilias, lupus erythematodes and alopecia greata.

A further subject of the present invention is the use of the compounds of the above formula for the prevention and treatment of allergic skin reactions, for example sun allergies (dermatitis solaris, dermatitis vernalis aurium, dermatitis bullosa pratensis), and food allergies, for example allergies triggered by bamboo shoots, bitter almonds, beans, oysters and dates.

A further subject of the present invention is the use of the compounds of the above formula for the treatment of acute skin injuries, for example burns (phototoxic reactions, photoallergic reactions, skin damage caused by ionizing radiation (acute and chronic radio-dermatitis)) including sunburn and other radiation damage, and for the treatment of skin injuries such as wounds and the resulting scars, and for striae cutis distensae and aging of the skin, such as wrinkling, including cellulitis, and cold injuries of the skin (congelation), skin injuries caused by the action of acids and alkalis, phacomatoses (Recklinghausen's disease), dermatomycoses, polymorphous photodermatoses, ichthyoses, and in the treatment of degenerate cells, such as in cancers, benign and/or malignant tumours.

In the context of the present invention, a distinction is to be made between treatment of diseases which have already developed and prevention in cases where there is a corresponding predisposition. Treatment of diseases means that, when they become manifest, treatment is given, which in the case of chronic diseases is generally permanent and, in the case of temporary diseases, takes place only during their occurrence.

Prevention means seeking to ensure that diseases do not occur. If, for example, an infant has a predisposition to chronic skin diseases such as psoriasis or neurodermatitis, it is possible, according to the invention, to provide preventive treatment using a pharmaceutical preparation containing the compound shown above. It has been found that children given preventive treatment during their infancy, which is when such skin diseases usually occur, did not develop the disease or developed only a very weak form of the disease, much less pronounced than that found in their parents.

The compounds of the formula I used according to the invention can be administered in any desired pharmaceutical presentation. For the use according to the invention, the compounds of the formula I are preferably administered topically. The compounds are preferably used in combination with excipients and diluents customary in the pharmaceutical industry for topical applications.

For the above use, the dose to be administered depends on the compound used, the type of administration and the type of treatment. Very good results are obtained if one or more compounds of the formula I are administered locally once or several times daily at a concentration of 1×10⁻⁴ to 1% by weight.

The presentations can contain the excipients and diluents customary in the pharmaceutical industry. Examples of suitable pharmaceutical forms are solutions, emulsions, suspensions, lotions, gels, ointments or creams.

The compounds of the formula I are preferably applied in combination with a synthetic or naturally occurring oil. The use of plant oils, in particular almond oil, peanut oil, sesame oil, olive oil, wheat-germ oil, maize-germ oil, thistle oil, soya oil, sunflower oil, coconut oil, avocado oil, palm kernel oil and cocoa butter, and also jojoba oil, evening primrose oil and calendula, has proven particularly suitable.

A further subject of the present invention is a pharmaceutical composition containing compounds of the formula I and a plant oil or fat.

In a further preferred embodiment of the present invention, the compounds used according to the invention are employed in combination with one or more further active substances which have a positive, protective and/or caring action on the skin. Examples of such substances are light filters, plant extracts, such as aloe vera or chamomile extracts, biopolymers, such as hyaluronic acid, chitin and collagen derivatives, constituents of the epidermal lipids, and vitamins and provitamins, for example panthenol and α-tocopherol. Other possible active substances which may be mentioned are hydroxycarboxylic acids, liposomes and niosomes or nanoparts.

The actions of the compounds of the formula I are evident from the following tests:

### EXAMPLES

Preparation of an ointment: 460 ml of avocado oil, 525 mg of cyanocobalamin, 175 mg of hydroxycobalamin, 80 g of emulsifier (methyl glycoside stearate), 2 g of potassium sorbate, 1 g of anhydrous citric acid depending on pH, mixed, and then made up to 1000 ml with distilled water.

1. Preventive Effect in Cases Where a Prediposition to Neurodermatitis is Inherited From the Parents

In a group of 10 infants (0-1 year) with parents, or at least one parent, suffering from manifest neurodermatitis, the ointment prepared in accordance with the above example was applied 3 times daily for a period of 3 months to the whole surface of the skin. The effect of the ointment according to the invention on the skin was monitored for 6 months.

During this monitoring period, no neurodermitic skin changes appeared in any of the infants studied.

2. Preventive Effect in Cases of Sun Allergy

In a group of 20 adults with a history of pronounced sun allergy, the ointment produced in accordance with the above example was applied once daily to the skin of the chest and arms for a period of one month prior to sun exposure in the case of 10 test subjects, and for a period of two months prior to sun exposure in the case of the remaining 10 test subjects.

Upon exposure to the sun, one patient from the group pre-treated for one month developed mild dermatitis solaris. The other test subjects showed normal tanning according to their skin type.

3. Effect on Damage Caused by Ionizing Radiation

In a group of 7 adults who were exposed to a radiation dose of between 40 and 60 Gy and had acute radiodermatitis, the damaged areas of skin were coated four times daily with the above-described ointment for a period of 14 days.

Complete remission was achieved in 5 patients. The subcutaneous fatty tissue showed a normal structure. Two patients still showed minor lesions, but in these cases too the improvement was clearly discernible, so that it can be assumed that continuation of the therapy beyond the 14 days leads to complete remission.

4. Effect on Burns

In a group of 25 patients with second-degree burns, the above-described ointment was applied twice daily during the first three days and thereafter once daily until complete healing. In one patient with third-degree burns of the right forearm, the area was treated twice daily for a period of 2 weeks, thereafter once daily until complete healing.

The treatment time for the patients with second-degree burns was dependent on the size of the damaged surface and was on average 7 days. The patient with the third-degree burns required 5 weeks of treatment until complete remission without scars.

5. Effect on Skin Cancer

In studies of in vitro and in vivo cells, a reversal of the proliferation of the degenerate cells could clearly be observed, and thus also a regression and cure in cases of skin cancer.

The reversal of the degenerate cells in cell cultures was approximately 25% in 2 weeks and approximately 70% in 4 weeks. In 7 cases, it was found in vivo that, after about 8 weeks, the manifestations were no longer detectable at the site of the initial manifestation.

From the above examples it will be evident that the use, according to the invention, of compounds of the formula I has a pronounced action both in preventive treatment and in the field of skin regeneration.

1. The use of corrinoids of the formula I 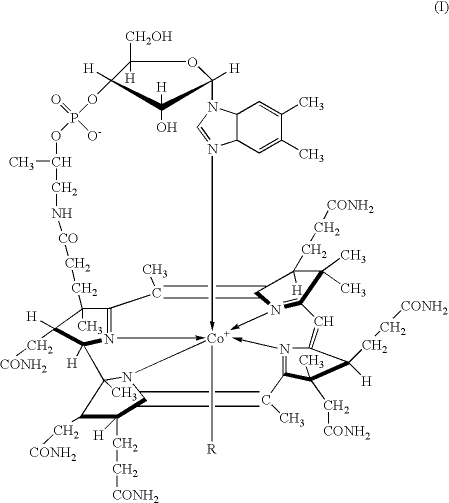
in which R stands for CN, OH, CH₃ or H₂O, for building up and improving the resistance of the skin.

2. The use according to claim 1, characterized in that the compounds of the formula I are used for the prevention of chronic skin diseases, in particular of inflammatory and hyperproliferative skin diseases, and cutaneous manifestations of immunologically induced diseases, for example psoriasis, atopic dermatitis, contact dermatitis and other eczematous dermatitides, seborrheic dermatitis, neurodermatitis, decubitus ulcers, lichen planus, pemphigus, bullous pemphigoid, epidermolysis bullosa, urticaria, angioedemas, vasculitis, erythemas, cutaneous eosinophilias, lupus erythematodes and alopecia greata.

3. The use according to claim 1, characterized in that the compounds of the formula I are used for the prevention and treatment of allergic skin reactions, in particular sun allergies (dermatitis solaris, dermatitis vernalis aurium, dermatitis bullosa pratensis), and food allergies, for example allergies triggered by bamboo shoots, bitter almonds, beans, oysters and dates.

4. The use according to claim 1, characterized in that the compounds of the formula I are used for the treatment of acute skin injuries such as cold injuries (congelation), skin injuries caused by the action of acids and alkalis, phacomatoses (Recklinghausen's disease), dermatomycoses, polymorphous photodermatoses, ichthyoses, and in the treatment of degenerate cells, such as in cancers, benign and/or malignant tumours.

5. The use according to claim 1, characterized in that the acute skin injuries are burns, including sunburn (dermatitis solaris, dermatitis vernalis aurium, dermatitis bullosa pratensis) and other radiation injuries (phototoxic reactions, photo-allergic reactions, skin damage caused by ionizing radiation (acute and chronic radiodermatitis)), wounds, and the resulting scars.

6. The use according to claim 1, characterized in that the compound of the formula I is used for treating manifestations of aging of the skin, such as senile elastosis, and in cases of striae cutis distensae, and wrinkling, including cellulitis.

7. The use according to one of claims 1 to 6, characterized in that customary excipients and/or diluents are also used.

8. The use according to one of claims 1 to 6, characterized in that one or more components having a positive, protective and/or caring action are additionally used.
